(19) United States
(12) Patent Application Publication
(10) Pub. No.: US 2018/0256114 A1
(43) Pub. Date: Sep. 13, 2018

(54) EXERCISE ASSISTANCE SYSTEM, INFORMATION DEVICE, MEASUREMENT DEVICE, EXERCISE ASSISTANCE METHOD, AND PROGRAM

(71) Applicant: SEIKO EPSON CORPORATION, Tokyo (JP)

(72) Inventors: Shinji HATTORI, Ina-shi (JP); Hironori HASEI, Azumino-shi (JP); Masakazu ISOMURA, Kai-shi (JP)

(73) Assignee: SEIKO EPSON CORPORATION, Tokyo (JP)

(21) Appl. No.: 15/756,400

(22) PCT Filed: Aug. 30, 2016
(86) PCT No.: PCT/JP2016/003933

§ 371 Date: Feb. 28, 2018

(30) Foreign Application Priority Data
Sep. 4, 2015 (JP) 2015-174447

(51) Int. Cl. A61B 5/00; A63B 24/00; A61B 5/083; A61B 5/024; A61B 5/11

(52) U.S. Cl. CPC A61B 5/7275; A63B 24/0062; A61B 5/0833; A63B 2024/0065; A61B 5/1118; A63B 2024/0068; A61B 5/02438

(57) ABSTRACT

An exercise assistance system that supports a user and acquires basic information. There is provided an exercise assistance system including an acquisition section that acquires measurement data including at least one of biological information and activity information of a user, a determination section that determines whether measurement period of the measurement data satisfies a predetermined condition, and a processing section that calculates basic habit information and basic physical strength information based on the measurement data in a case where the determination section satisfies the predetermined condition, and generates first information to notify the user based on the measurement data in a case where the determination section does not satisfy the predetermined condition.

### TECHNICAL FIELD

The present invention relates to an exercise supporting system, an information device, a measurement device, an exercise supporting method, and a program.

### BACKGROUND ART

In the related art, PTL 1 discloses an exercise supporting system that generates exercise advice for a user using measurement data measured by a measurement terminal worn on the user for measuring an exercise amount and provides the advice to the user. The system analyzes a basic exercise situation of the user using the measurement data for a certain period and determines an exercise plan to be worked after the certain period.

### CITATION LIST

### Patent Literature

PTL 1: JP-A-2001-286446

### SUMMARY OF INVENTION

### Technical Problem

However, in the exercise supporting system disclosed in PTL 1, there is a possibility that the user may feel bored or uneasy for the certain period of measuring the measurement data. In the system, no feedback is made to the user for the certain period such as several days to one week. For the reason, the measurement for the certain period is left to the user, and there is a possibility that a user, particularly, who first participates in an exercise supporting service by the system may feel uneasy about the exercise supporting service for the certain period.

Even though the measurement data for the certain period is data for analyzing the basic exercise situation (exercise habit and exercise ability) of the user, there is a tendency for the user to exercise harder than a usual exercise. In an exercise plan determined based on such measurement data, there is a possibility that it becomes a plan with high barriers far from a usual exercise situation of the user and hinders the continuation of exercise of the user.

### Solution to Problem

The invention is made to solve at least a part of the problems described above and can be realized as the following forms or application examples.

### APPLICATION EXAMPLE 1

An exercise supporting system according to this application example includes an acquisition section that acquires measurement data including at least one of biological information and activity information of a user, a determination section that determines whether measurement period of the measurement data satisfies a predetermined condition, and a processing section that calculates basic habit information and basic physical strength information based on the measurement data in a case where the determination section satisfies the predetermined condition, and generates first information to notify the user based on the measurement data in a case where the determination section does not satisfy the predetermined condition.

According to the application example, the processing section generates the first information in the case where the measurement period of the measurement data of the user does not satisfy the predetermined condition. Since the first information is the information for notifying the user, it is the information that can be fed back to the user during the measurement period. Accordingly, in the exercise supporting system according to the application example, there is a reduced possibility that the user who first uses the exercise supporting service feels uneasy with respect to the exercise supporting service during the certain period.

Since the first information is generated based on the measurement data, it can be information in which the exercise situation of the user during the measurement period is reflected. For example, in a case where there is a possibility that the user performs exercise far from usual, it is possible to generate the first information that prompts the user to exercise as usual. Accordingly, it is possible to create an exercise plan based on the exercise situation of the user as usual.

### APPLICATION EXAMPLE 2

The predetermined condition according to the application example described above is satisfied in a case where the measurement period exceeds the predetermined number of days and is not satisfied in a case where the measurement period is equal to or less than the predetermined number of days.

According to the application example, in the case where the measurement period exceeds the predetermined number of days, it is possible to analyze a habitual exercise situation in the predetermined number of days. In the case where the measurement period is equal to or less than the predetermined number of days, it is possible to generate the first information including the number of remaining days for the user.

### APPLICATION EXAMPLE 3

The first information according to the application example described above includes at least one of an advice display screen, a chat screen, and a countdown display screen.

According to the application example, since it is possible to provide advice about the exercise situation of the user, exchange the information with the user by a chat, and display the countdown on the number of remaining days of the measurement period during the measurement period, there is a less possibility that the user feels bored and uneasy.

### APPLICATION EXAMPLE 4

The determination section according to the application example described above further compares a data amount of the measurement data with a predetermined amount in the case where the predetermined condition is satisfied, and the predetermined condition is satisfied in a case where the data amount is equal to or larger than the predetermined amount and the predetermined condition is not satisfied in a case where the data amount is less than the predetermined amount.

According to the application example, it is possible to calculate the basic habit information and the basic physical strength information representing the habitual exercise situation of the user by the data amount equal to or larger than the predetermined amount.

### APPLICATION EXAMPLE 5

The basic habit information according to the application example described above includes at least one of an exercise time, the number of days of exercise, calorie intake, and calorie consumption.

According to the application example, it is possible to calculate the habitual exercise time, the number of days, the exercise amount (calorie consumption), and a meal amount (calorie intake) as the basic habit information.

### APPLICATION EXAMPLE 6

The basic habit information according to the application example described above is an average value or a sum value per certain period in the measurement period.

According to the application example, for example, it is possible to set a goal based on the basic habit information in the exercise plan for each certain period using the average value or the sum value of the basic habit information in the certain period.

### APPLICATION EXAMPLE 7

The basic physical strength information according to the application example described above includes at least one of a physical strength level, maximum oxygen uptake, an average heart rate during exercise, a rising heart rate during exercise, and a heart rate during rest.

According to the application example, since the physical strength level and the exercise ability of the user are indexed, it is possible to accurately capture changes in the physical strength level and the exercise ability during exercise.

### APPLICATION EXAMPLE 8

The exercise supporting system according to the application example described above further includes a measurement terminal having a measurement section that measures the measurement data of the user and an output section that outputs the measurement data to the acquisition section.

According to the application example, it is possible to select and measure the measurement data used by the determination section and the processing section by including the measurement terminal.

### APPLICATION EXAMPLE 9

An information device includes a display section that displays the first information generated by the processing section of the exercise supporting system according to the application example described above.

According to the application example, it is possible to notify the user using the information device of the first information.

### APPLICATION EXAMPLE 10

A measurement device includes an output section that outputs the measurement data to the acquisition section of the exercise supporting system according to the application example described above, a measurement section that measures the measurement data of the user, and a display section that displays the first information generated by the processing section.

According to the application example, it is possible to measure the measurement data of the user using the measurement device and notify the user of the first information.

### APPLICATION EXAMPLE 11

An exercise supporting method according to the application example includes an acquisition step of acquiring measurement data including at least one of biological information and activity information of a user, a determination step of determining whether a measurement period of the measurement data satisfies a predetermined condition, and a processing step of calculating basic habit information and basic physical strength information based on the measurement data in a case where the determination step satisfies the predetermined condition, and generating first information to notify the user based on the measurement data in a case where the determination step does not satisfy the predetermined condition.

According to the application example, in the processing step, the first information is generated in the case where the measurement period of the measurement data of the user does not satisfy the predetermined condition. Since the first information is the information for notifying the user, it is the information that can be fed back to the user during the measurement period. Accordingly, in the exercise supporting method according to the application example, there is a reduced possibility that the user who first uses the exercise supporting service feels uneasy with respect to the exercise supporting service during the certain period.

Since the first information is generated based on the measurement data, it can be information in which the exercise situation of the user during the measurement period is reflected. For example, in a case where there is a possibility that the user performs exercise far from usual, it is possible to generate the first information that prompts the user to exercise as usual. Accordingly, it is possible to create an exercise plan based on the exercise situation of the user as usual.

### APPLICATION EXAMPLE 12

A program according to the application example causes a computer to execute an exercise supporting method including an acquisition step of acquiring measurement data including at least one of biological information and activity information of a user, a determination step of determining whether a measurement period of the measurement data satisfies a predetermined condition, and a processing step of calculating basic habit information and basic physical strength information based on the measurement data in a case where the determination step satisfies the predetermined condition, and generating first information to notify the user based on the measurement data in a case where the determination step does not satisfy the predetermined condition.

By causing the computer to execute the program according to the application example, the processing step generates the first information in the case where the measurement period of the measurement data of the user does not satisfy the predetermined condition. Since the first information is the information for notifying the user, it is the information that can be fed back to the user during the measurement period. Accordingly, by executing the program according to the application example, there is a reduced possibility that the user who first uses the exercise supporting service feels uneasy with respect to the exercise supporting service during the certain period.

Since the first information is generated based on the measurement data, it can be information in which the exercise situation of the user during the measurement period is reflected. For example, in a case where there is a possibility that the user performs exercise far from usual, it is possible to generate the first information that prompts the user to exercise as usual. Accordingly, it is possible to create an exercise plan based on the exercise situation of the user as usual.

### BRIEF DESCRIPTION OF DRAWINGS

FIG. 1 is an explanatory diagram representing an outline of an exercise supporting system.

FIG. 2 is a diagram illustrating a flow of an exercise supporting service.

FIG. 3 is a block diagram showing a schematic configuration of the exercise supporting system.

FIG. 4 is a diagram showing an example of an advice display screen.

FIG. 5 is a diagram showing an example of a countdown display screen.

FIG. 6 is a diagram showing an example of a chat screen.

FIG. 7 is a diagram showing an example of a basic information display screen.

FIG. 8 is a diagram showing an example of a goal presentation screen.

FIG. 9 is a flowchart diagram showing a flow of basic information acquisition processing.

FIG. 10 is an explanatory diagram representing an outline of a measurement device according to a modification example.

### DESCRIPTION OF EMBODIMENTS

Hereinafter, embodiments of the invention will be described with reference to drawings. In each of the following drawings, in order to make each section and each screen a recognizable size, a scale of each section and each screen is different from an actual scale.

### Embodiment 1

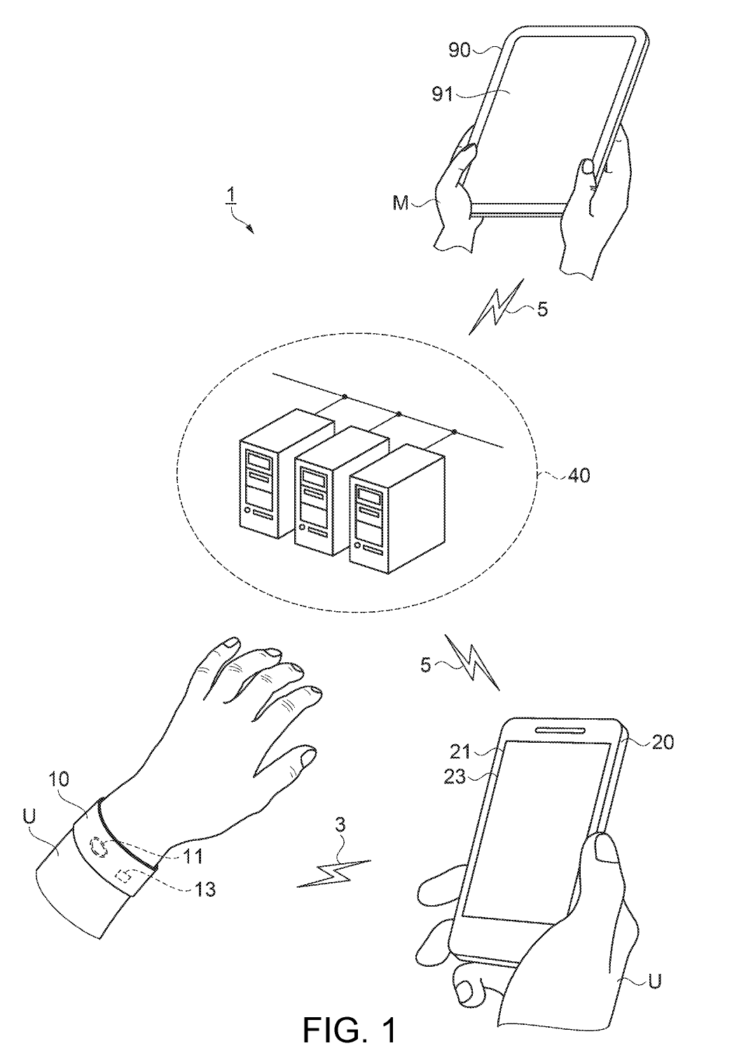

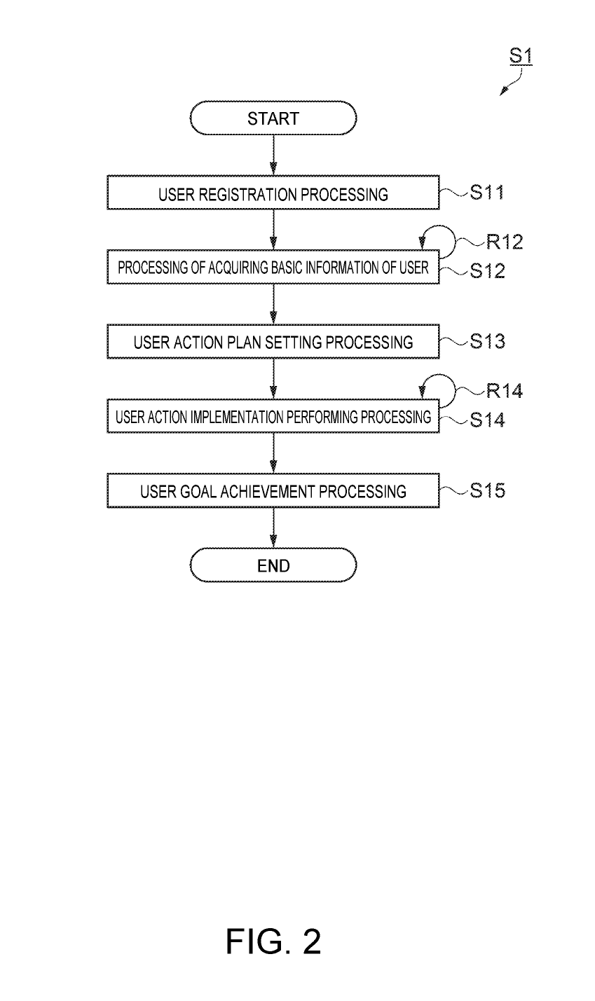

FIG. 1 is an explanatory diagram representing an outline of an exercise supporting system. FIG. 2 is a diagram illustrating a flow of an exercise supporting service. First, the outline of the exercise supporting system according to an embodiment 1 will be described.

### (Outline of Exercise Supporting System)

The exercise supporting system 1 (FIG. 1) is a system for realizing a service of an exercise supporting service S1 (FIG. 2). The exercise supporting service S1 is the service for supporting an improvement action of a user U who is suffering from a disease caused by a life habit or a user U who desires to improve an exercise habit and a dietary habit.

In the exercise supporting system 1, support for the improvement action of the user U is realized by using experience and knowledge of a mentor M who is familiar with health guidance and dietary guidance. The exercise supporting system 1 is configured to have a measurement terminal 10 worn on the body of the user U, an information device 20 to be used, a mentor device 90 used by the mentor M, a server 40, and the like. The server 40 is configured to have a plurality of server functions such as a web server, a database server, and an application server. The server 40 includes a function of acquiring biological information and activity information from the measurement terminal 10 and the information device 20 and accumulating the pieces of information as measurement data, a function of generating a web page screen or the like displayed on the information device 20 or the mentor device 90, and the like.

Here, an outline of the exercise supporting service S1 will be described with reference to FIG. 2. The exercise supporting service S1 sequentially proceeds processing from processing S11 to processing S15 in order to support the improvement action of the user U and contribute to goal achievement of the user U.

The processing S11 is user registration processing. The processing generates a web page for user registration and acquires unique information relating on the user U, purpose information, service period information, and the like. The unique information is information such as the name, age, gender, occupation, current health condition, past medical history of the user U. The purpose information is information on a purpose of using the exercise supporting service S1 such as a weight loss, a desire to reduce body fat, and an increase in physical strength. The service period information is a contract period in which the user U can receive the service such as two weeks, one month, and three months. In processing S11, a mentor M who supports the user U is determined. In processing S11, there may be included processing of lending the measurement terminal 10 to the user U or processing of allowing the user U to designate the mentor M who supports the user U.

The processing S12 is processing of acquiring basic information of the user. An action plan is set before the improvement action by the user U is started in earnest. Therefore, in order to design the action plan, information such as basic physical strength and a basic habit relating on a meal and exercise of the user U is collected.

The measurement terminal 10 is worn always on the user U, and the measurement data can be measured while living life as usual.

The measurement data is transmitted from the measurement terminal 10 to the server 40. The server 40 receives and accumulates the measurement data. The server 40 also receives and accumulates data such as meal content (calorie intake) not measured by the measurement terminal 10 from the information device 20.

The server 40 receives the data every day from the measurement terminal 10 and the information device 20. The server 40 transmits information for following the user every day to the information device 20. The information specifically includes advice information for the user U, chat information exchanged between the user U and the mentor M, countdown information for notifying the user U of the number of remaining days of a measurement period, and the like.

Repeat R12 indicates that the processing is repeated every day for a predetermined number of days (preferably seven days). Basic physical strength information and basic habit information are calculated after the predetermined number of days. The basic physical strength information is information representing the basic physical strength of the user U such as a physical strength level, a maximum oxygen uptake, and an average heart rate during exercise. The basic habit information is information representing the exercise habit of the user U such as daily exercise time, regular exercise time per week, and calorie intake and calorie consumption per week.

The processing S13 is user action plan setting processing. A plan for the improvement action of the user U is created based on the basic physical strength information and the basic habit information calculated in processing S12. In the plan for the improvement action, when information on the exercise habit and the dietary habit of the user U is acquired from the basic habit information, a plan of gradually increasing an exercise amount or reducing a meal amount is created based on the acquired information. A basic habit of the user U cannot be changed easily, and consideration is made to avoid that a forceful change of the basic habit for approaching a target makes it difficult to continue the improvement action.

When the action plan corresponding to the exercise habit, the dietary habit, or the like of the user U is determined, a plan relating on the increase in physical strength of the user U is determined. In the plan, an exercise load, a meal restriction amount, and the like are determined such that a change from the basic physical strength information to physical strength information in a goal achievement state gradually occurs. As described above, the action plan of the user U is determined based on the basic physical strength information and the basic habit information. The plan for the improvement action of the user U may be performed by the mentor M or implemented by the server 40. Alternatively, the user U and the mentor M may discuss and determine the plan using a chat function.

The processing S14 is user action implementation performing processing. The processing is processing of supporting the user U who acts according to the action plan (action such as exercise and meal restriction). Specifically, in server 40, an analysis of whether a result as planned is achieved is performed based on the information transmitted from the measurement terminal 10 and the information device 20. The server 40 provides the advice information and various pieces of information on a good or bad action and an improvement for the goal achievement while adding information obtained by the mentor M based on an analysis result to the user U. Repeat R14 indicates that the processing is repeated for each certain period (for example, one week) for a period (for example, three months) from a goal setting to the goal achievement. In server 40, work of modifying the plan itself is also performed as necessary.

The processing S15 is user goal achievement processing. The processing is processing in a case where a final goal of the user U is achieved or processing in a case where a service providing period of a service selected by the user ends in processing S14. An evaluation for the user U and an evaluation for the mentor M are performed, and a series of the exercise supporting services S1 for the user U ends.

As described above, in the exercise supporting service S1, the processing from S11 to S15 is processed sequentially to support the improvement action of the user U actively and carefully, thereby increasing a success rate of the goal achievement of the user U.

The inventor actually operates a similar exercise supporting system in the past and performs an analysis based on many failure cases that cannot continue an improvement action. Thus, the inventor finds that it is important to collect measurement data of the user U as usual (processing S12) in order to continue the improvement action of the user U and succeed in achieving a final goal.

In the embodiment, the processing of acquiring the basic information of the user which is a feature of the invention (processing S12) will be described mainly for the exercise supporting system 1. The detail description of the processing of S11, S13, S14, and S15 realized in the exercise supporting system 1 will be omitted.

### (Configuration of Exercise Supporting System)

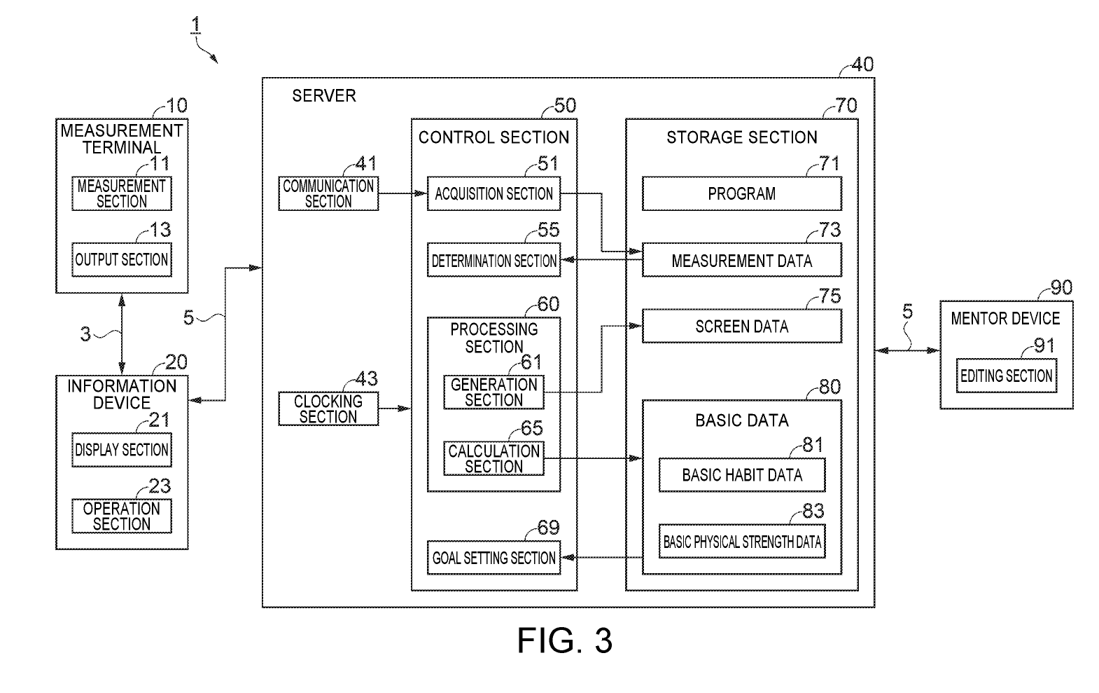

FIG. 3 is a block diagram showing a schematic configuration of the exercise supporting system.

The exercise supporting system 1 is configured to have the measurement terminal 10, the information device 20, the server 40, the mentor device 90, and the like.

The measurement terminal 10 and the information device 20 are connected by a communication 3. The server 40 is connected to the information device 20 and the mentor device 90 by a communication 5.

The measurement terminal 10 is a small wristwatch type apparatus which is worn on or carried by the body of the user U and is configured to have a measurement section 11, an output section 13, a control section (not shown) for controlling the measurement section 11 and the output section 13, a storage section (not shown) for storing data and a processing program, a clocking section (not shown) including a real-time clock, and the like.

The measurement section 11 includes a detection sensor (not shown) for detecting a signal relating on the biological information and the activity information of the user U and a function of calculating measurement data from the detected detection signal. The detection sensor is, for example, a sensor unit including a pulse wave sensor, an acceleration sensor, a gyro sensor, a global positioning system (GPS) sensor (none of them are shown). In each sensor unit, the biological information such as a pulse rate, the calorie consumption, and exercise intensity and the activity information are calculated as the measurement data by a known method from the detected detection signal. The measurement data is calculated for each unit time (for example, one second) and is stored in the storage section together with measured time information.

The output section 13 is a communication adapter having a function of reading the measurement data from the storage section and outputting (transmitting) the read measurement data to another device including the information device 20. The output section 13 reads the measurement data from the storage section using a signal from the clocking section or another device as a trigger and establishes communication with the other devices. When the communication is established, the measurement data is transmitted to another device in accordance with a communication protocol. The communication adapter is a wireless LAN adapter as a preferable example and has an internet protocol (IP) and the communication protocol common to an external device. The invention is not limited to the configuration. A communication adapter that can communicate wirelessly may be used and, for example, a Bluetooth (registered trademark) adapter may be used. The output section 13 transmits the measurement data to the information device 20 through the communication 3. The measurement data transmitted to the information device 2 is acquired by the server 40 (acquisition section 51) through the communication 5. The output section 13 may be configured to establish the communication with the server 40 (communication section 41) and directly transmit the measurement data from the measurement terminal 10 to the server 40.

The output section 13 may be configured to include a physical communication terminal, be connected to another device through a cable, and transmit the measurement data.

The information device 20 is a general smartphone, a tablet terminal, a personal computer (PC), or the like and configured to have a display section 21, an operation section 23, a communication section (not shown), a clocking section (real-time clock), an imaging section that captures an image, a storage section that stores measurement data and a program, a control section that controls each section, and the like.

The display section 21 is a function section having a display apparatus such as a liquid crystal display (LCD), a display control section, and a web browser function and displays character information, image information, and the like received from the server 40 or the mentor device 90 through the communication 5.

The operation section 23 is an input apparatus configured to have a button switch, a touch panel, and the like.

The communication section is a communication adapter that can communicate with the measurement terminal 10 through the communication 3 and a communication adapter that can communicate with the server 40 and the mentor device 90 through the communication 5.

The control section controls the communication section to communicate with the measurement terminal 10 and displays an operation state of the measurement terminal 10 on the display section 21. For example, a change in the heart rate of the user U is displayed on the display section 21 as a graph. The control section transmits a signal of a measurement data transmission request to the measurement terminal 10 at a timing of establishing communication with the measurement terminal 10 and receives the measurement data from the measurement terminal 10. The received measurement data is stored in the storage section. The control section establishes communication with the server 40 preferably with frequency of once or more times per day and transmits the measurement data accumulated in the storage section to the server 40.

Further, the control section controls the display section 21 and the operation section 23, acquires meal information from the user U, and calculates the calorie intake. The calculation of the calorie intake from the meal information is calculated by a known method. For example, a method in which meal components from an image of a meal captured by the imaging section are estimated and an energy amount for each meal component is integrated and calculated as the calorie intake, a method in which the user U selects a food in the meal content and a meal amount from the options by controlling the operation section 23 and an energy amount of the food is calculated, or the like is preferable. The control section transmits information on the calorie intake in addition to the measurement data to the server 40.

The mentor device 90 is a general smartphone, a tablet terminal, a PC, or the like and configured to have an editing section 91, a communication section (not shown), a clocking section (real-time clock), a storage section, a control section that controls each section, and the like.

The editing section 91 is a function section that includes a display section and an operation section and can edit the character information and the image information. The editing section 91 can perform various types of editing such as an advice wording for the user U, a chat wording, an attachment of an image, a link of reference information. Edited information is transferred to the server 40 through a protocol or the like such as hypertext transfer protocol (HTTP). The server 40 generates a web screen or the like using the edited information.

The communication section is a communication adapter that can communicate with the server 40 and the information device 20 through the communication 5.

The server 40 is a general PC or a server apparatus and is configured to have a communication section 41, a clocking section 43, a control section 50, a storage section 70, and a display section such as an LCD (not shown), an operation section such as a keyboard and a mouse. The server 40 may have a plurality of server functions such as the web server, the database server, and the application server as described above and may be configured by a plurality of PCs and servers.

The communication section 41 is a network adapter and is connected so as to be able to transmit and receive various data to and from the information device 20 and the mentor device 90 and the server 40. In a case where the server 40 is configured by the plurality of servers, the plurality of PCs are connected such that various data can be transmitted and received by an intranet or the internet.

The clocking section 43 is a real-time clock and has, for example, a clocking function such as a calendar function and a clock function. The clocking such as the measurement period and the predetermined number of days is performed.

The control section 50 is configured to have a processor such as a central processing unit (CPU) and controls each section such as the communication section 41, the clocking section 43, the storage section 70 that configure the server 40 according to various programs including a program 71 stored in the storage section 70.

The control section 50 has an acquisition section 51, a determination section 55, a processing section 60, and a goal setting section 69 as function sections. However, the function sections are described only as an example, and all the function sections and the configurations may not be an essential configuration. A function section other than the function sections may be the essential configuration.

### (Acquisition Section)

The acquisition section 51 has a function of acquiring the measurement data. The measurement data measured by the measurement terminal 10 is accumulated in the information device 20. When an accumulation amount reaches to a certain extent (preferably, measurement data amount for one day at most), the information device 20 establishes communication with the communication section 41 of the server 40. With a trigger of the communication section 41, the acquisition section 51 receives the measurement data from the information device 20 and stores the received data in the storage section 70 as measurement data 73.

In the measurement data 73, information on a measurement time, the pulse rate, the calorie consumption, the exercise intensity, the calorie intake, and the like is stored in time series, for example, in units of one second.

### (Determination Section)

The determination section 55 determines whether the measurement period satisfies a predetermined condition. The determination section 55 acquires a measurement time of the measurement data initially measured by the user U from the measurement data 73. Thereafter, a difference from a clocking time measured by the clocking section 43 is taken and the measurement period is counted.

In the determination section 55, for example, two type conditions are defined. A first type condition is a condition in which it is determined that the condition is satisfied in a case where the measurement period described above exceeds the predetermined number of days and it is determined that the condition is not satisfied in a case where the measurement period is equal to or less than the predetermined number of days. A second type condition is a condition in which it is determined that the condition is satisfied in a case where the first type condition is satisfied and further a data amount of the measurement data is equal to or larger than a predetermined amount, and it is determined that the condition is not satisfied in a case where the data amount is less than the predetermined amount. The defined two type conditions can be selected as either the first type condition only or the first type condition and the second type condition. Which condition is selected differs for each user or each group or organization to which a target user belongs and the condition is stored in advance in the storage section 70.

Here, the first type condition and the second type condition will be exemplified and described.

The predetermined number of days of the first type condition is seven days (one week) as a preferable example. A period of a general life cycle is one week in which a holiday appears in a constant period. There is a high possibility that the basic habit information on action such as habitual exercise and meal of the user U can be obtained by setting the predetermined number of days to one week.

The predetermined amount of the second type condition is a sum of the data amount measured every day for the predetermined number of days (seven days) as a preferable example. In a case where the measurement data for several days of the predetermined number of days is missing, it is difficult to obtain the basic habit information and the basic physical strength information of the user U. Therefore, the conditions are defined.

### (Processing Section)

The processing section 60 is configured by function sections of the generation section 61 and the calculation section 65 which are processed based on a determination result by the determination section 55.

The generation section 61 is a function section executed in a case where a determination by the determination section 55 does not satisfy the predetermined condition and has a function of generating screen information provided to the user U. The measurement terminal 10 continues to measure the measurement data during the time.

The generation section 61 generates screen information for prompting the user U to continue measuring the measurement data. Screen information for guiding the user U so as to perform daily life as usual and the habitual exercise as described in processing S12 is also generated.

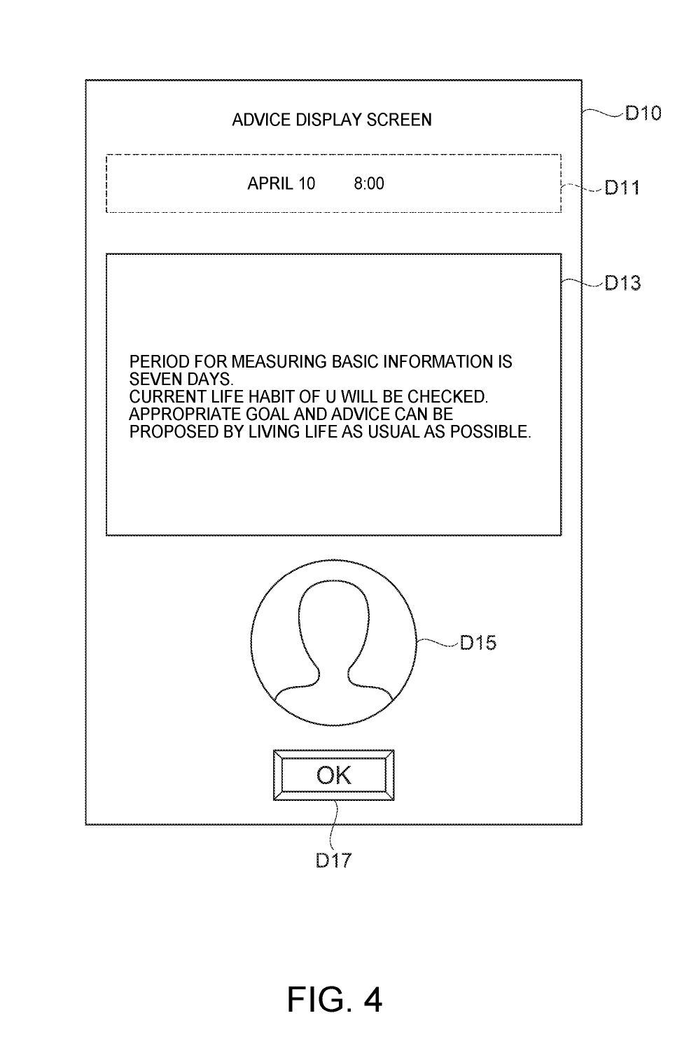

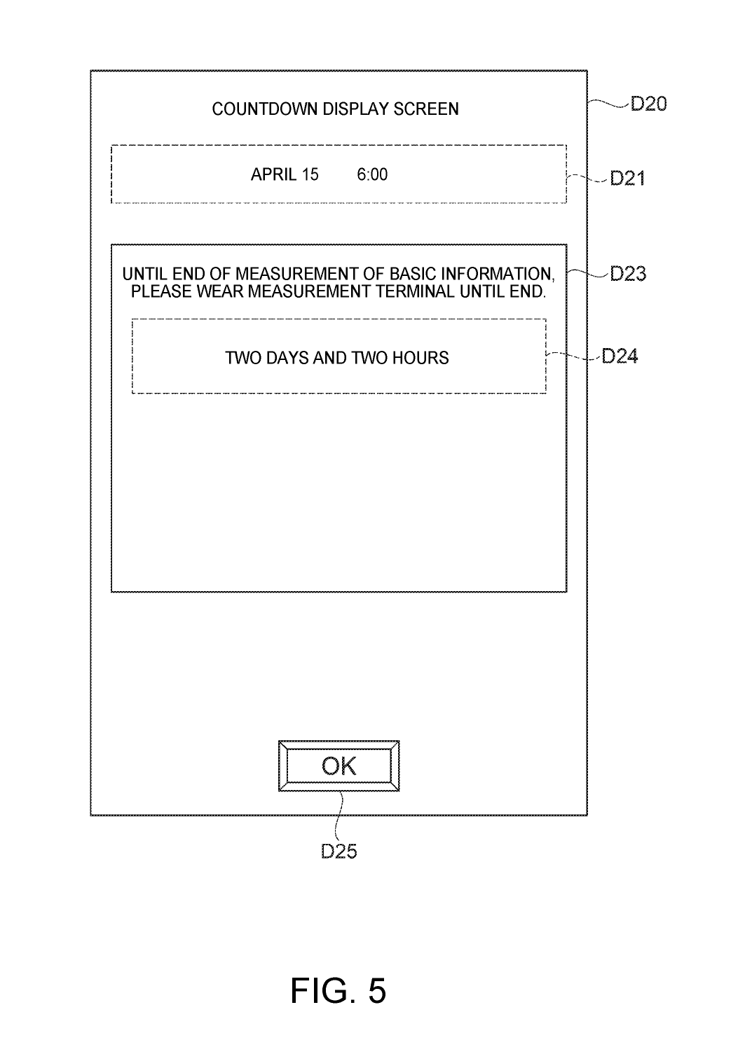

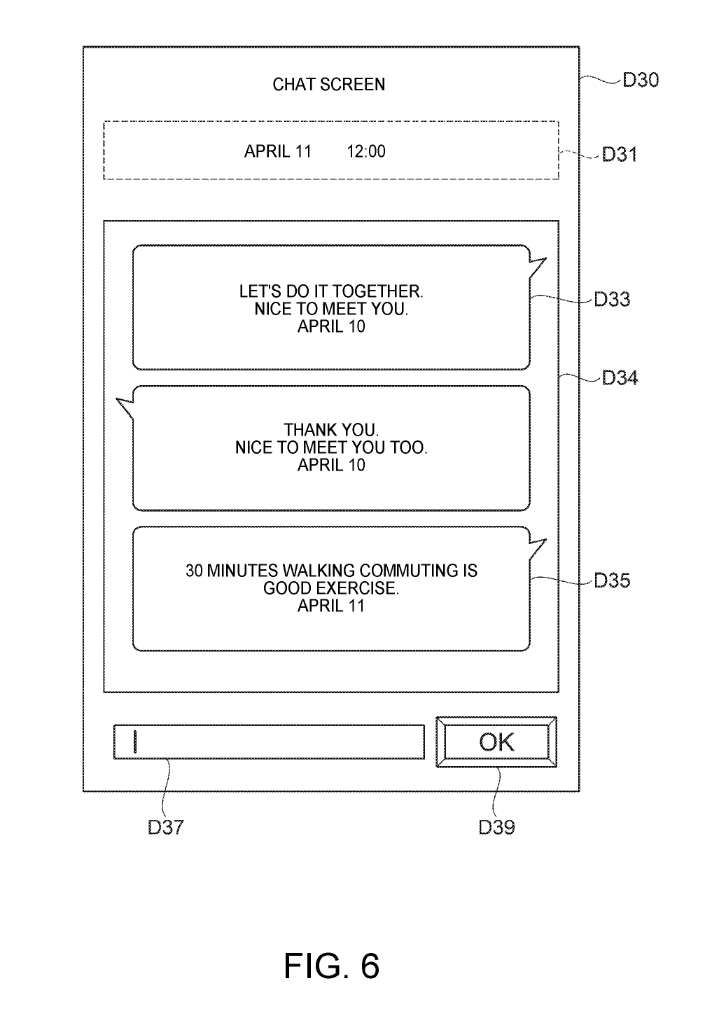

The screen information generated by the generation section 61 will be described. FIG. 4 is a diagram showing an example of an advice display screen, FIG. 5 is a diagram showing an example of a countdown display screen, and FIG. 6 is a diagram showing an example of a chat screen. An advice display screen D10, a countdown display screen D20, a chat screen D30 can be displayed and partially edited in a web browser or an application of the information device 20 and the mentor device 90.

However, the pieces of screen information are described only as an example, and all the pieces of screen information may not be the essential configuration. Screen information other than the pieces of screen information may be the essential configuration.

The screen information corresponds to first information.

The advice display screen D10 is a screen for displaying the advice information for the user U from the mentor M and is configured to have date and time information D11, an advice wording D13, a mentor screen D15, an OK button D17, and the like.

The advice display screen D10 is the screen which is generated by the generation section 61, then edited by the mentor M in the mentor device 90, and completed. The completed advice display screen D10 is displayed on the information device 20, and display content is notified to the user U.

The date and time information D11 is a region for displaying current date and time, and a date and time acquired from the clocking section of the device (information device 20, mentor device 90, and server 40) on which the advice display screen D10 is displayed is displayed and updated every minute.

The advice wording D13 displays an advice wording which the mentor M creates or edits. The mentor M checks the measurement data 73 to which new measurement data is added and creates a timely advice wording according to a latest situation of the user U. In an example of the wording, “A period for measuring basic information is seven days. Current life habit of U will be checked. An appropriate goal and advice can be proposed by living life as usual as possible.” is displayed. According to the wording example, the measurement of the measurement data of the user U is just started. Therefore, there is the advice wording that the period for measuring the basic information is seven days and the user lives as usual.

The mentor screen D15 is a face photograph image or an image of the mentor M. It is preferable to display an image with a smiley expression as an image to be displayed. An image in which the expression is changed may be displayed according to content of an advice wording.

The OK button D17 is a button for switching the display of the advice display screen D10 to another screen.

The countdown display screen D20 is a screen for displaying the number of remaining days and remaining times in the measurement period for the user U and is configured to have date and time information D21, countdown information D23, a countdown time D24, an OK button D25, and the like. The countdown display screen D20 is generated by the generation section 61, then displayed on the information device 20, and provided to the user U.

The date and time information D21 is a region for displaying current date and time, and a date and time acquired from the clocking section of the device (information device 20, mentor device 90, and server 40) on which the countdown display screen D20 is displayed is displayed and updated.

In the countdown information D23, the countdown time D24 is included, and a remaining day and time until the end of the measurement of the basic information (measurement data) and a fixed-form message “please wear measurement terminal until the end.” are displayed.

The countdown time D24 displays a calculated remaining day and time. The generation section 61 takes a difference between the measurement time of the measurement data initially measured by the user U acquired from the measurement data 73 and current date and time displayed in the date and time information D21, and calculates the remaining day and time.

The OK button D25 is a button for switching the display of the countdown display screen D20 to another screen.

The chat screen D30 is a screen for talking on a bulletin board between the user U and the mentor M, and is configured to have date and time information D31, chat wordings D33, D34, and D35, a wording input region D37, a transmission button D39, and the like.

The date and time information D31 is a region for displaying current date and time, and a date and time acquired from the clocking section of the device (information device 20, mentor device 90, and server 40) on which the chat screen D30 is displayed is displayed and updated.

The chat wordings D33 and D35 are chat wordings input by the mentor M.

The chat wording D34 is a chat wording input by the user U.

The wording input region D37 is a region for inputting a chat wording, and a character string input on a character input screen (not shown) is displayed.

In the transmission button D39, the character string displayed in the wording input region D37 is confirmed and displayed as the chat wording.

The advice display screen D10, the countdown display screen D20, and the chat screen D30 described above are only examples for exemplifying the invention, and some display regions may be deleted and another information may be added. For example, a region for displaying information on sleep and information on an imbalance and a tendency of the meal content may be added.

### (Calculation Section)

The calculation section 65 is realized in a case where the determination by the determination section 55 satisfies the predetermined condition and has a function of calculating the basic physical strength information and the basic habit information based on the measurement data.

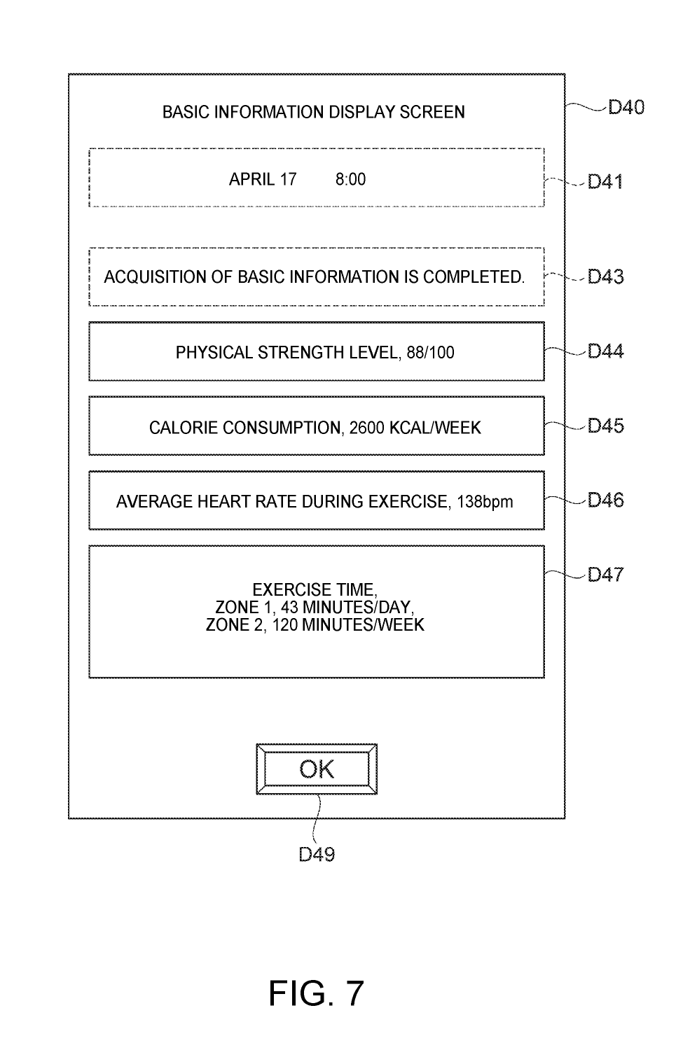

The calculation section 65 sequentially reads information on the pulse rate for the predetermined number of days, the calorie consumption, the exercise intensity, the calorie intake from the measurement data 73 and calculates the basic habit information and the basic physical strength information. The calculated basic habit information and basic physical strength information are stored in the storage section 70 as basic habit data 81 and basic physical strength data 83 of basic data 80, and a basic information display screen D40 (FIG. 7) on which information on the basic habit information and the basic physical strength information is posted is generated.

The basic habit information is information determined based on the exercise time, the number of days of exercise, the calorie intake, the calorie consumption, and the like.

In the calculation of the exercise time and the number of days of exercise, first, it is necessary to distinguish an exercise state from a non-exercise time (for example, normal state or sleeping state) and extract the exercise state. Therefore, it is possible to use the exercise intensity and the pulse rate acquired by the measurement terminal 10. Further, it is possible to derive a level of the exercise state (for example, intense exercise or light exercise) or the like by using the exercise intensity and the pulse rate.

For example, a light exercise is defined as three METS or more and less than six METS using exercise intensity (METS). A time zone of the light exercise is referred to as zone 1. Similarly, an intense exercise is defined as, for example, six METS or more and the time zone of the intense exercise is referred to as zone 2. A time of zone 1 and a time of zone 2 per day can be calculated for each zone.

For example, in a case where the pulse rate is used, the time of zone 1 and zone 2 per day is calculated in a case of setting zone 1 to a pulse rate (for example, 110 bpm or more and less than 150 bpm) slightly higher than the normal state and zone 2 to a pulse rate (150 bpm or more) higher than zone 1.

As described above, a sum of zone 1 per day is calculated as an exercise time per day. The number of days of exercise per week is calculated as the number of days of exercise by counting days in which the time of zone 2 is continuously one hour or more in one week.

The exercise time is an index of daily habitual exercise such as walking for 30 minutes on foot every day as the exercise habit.

The number of days of exercise is an index of weekly exercise habit such as going to a training gym twice or three times a week or jogging.

The calorie intake is calculated as a sum of calorie intake for one week acquired as the measurement data.

A sum of the calorie consumption per week may be calculated in order to analyze a balance with the calorie intake as the exercise habit.

In the period during which the basic habit information described above is calculated, per day or per week corresponds to per certain period. The certain period is not limited to one day and one week and may be another period as long as it is determined in advance. The calculation of the basic habit information described above is described as the sum (sum value), but may be an average value.

The basic habit information of the user is determined based on the information on the exercise time, the number of days of exercise, the calorie intake, the calorie consumption, and the like. Various determination methods can be considered. The basic habit information of the user U may be determined based on only any information of the exercise time, the number of days of exercise, the calorie intake, and the calorie consumption. A plurality of pieces of information of the exercise time, the number of days of exercise, the calorie intake, the calorie consumption are weighted, then scores are determined by addition processing, and the basic habit information of the user U may be determined based on the score. Further, the determination may be made in consideration of exercise frequency, exercise intensity distribution, and the like.

The basic physical strength information is information determined based on the maximum oxygen uptake, the average heart rate during exercise, a rising heart rate during exercise, a heart rate during rest, and the like. It is also possible to add an index of the physical strength level as the basic physical strength information using the pieces of information.

The maximum oxygen uptake is an index representing an aerobic exercise ability. The maximum oxygen uptake can be calculated by a known method using the pulse rate, the heart rate, and the activity information. For example, in JP-A-2014-195711, a method of calculating an accurate maximum oxygen uptake while correcting an initial maximum oxygen uptake estimated using the biological information and the activity information is disclosed. It is possible to estimate the maximum oxygen uptake of an object person (user U) by deriving an exercise habit level from the biological information and the activity information and substituting the derived level to a known Jackson equation (refer to Jackson A S, Blair S N, Mahar M T, Wier L T, Ross R M, Stuteville J E: Prediction of functional aerobic capacity exercise testing. Med Sci Sports Exerc 1990, 22: 863-870). In general, the pulse rate and the heart rate are the same unless a pulse defect such as arrhythmia occurs.

The average heart rate during exercise is an index correlated with a cardiopulmonary ability and muscular strength and is a value obtained by calculating the average heart rate for each level of the exercise intensity. For example, it is possible to calculate an average heart rate during the intense exercise by calculating an average pulse rate corresponding to zone 2 of the exercise intensity described above.

The rising heart rate during exercise is an index correlated with the cardiopulmonary ability and the muscular strength. For example, in a case where the cardiopulmonary ability is high, the rising heart rate during exercise slowly increases, and in a case where the cardiopulmonary ability is low, the rising heart rate rapidly increases. The rising heart rate during exercise is calculated using a change amount of the exercise intensity and a change amount of the pulse rate per unit time (for example, one second).

The heart rate during rest correlates with age and the cardiopulmonary ability. For example, in the same age, as the cardiopulmonary ability is higher, the heart rate during rest tends to be lower. There is a personal difference in the heart rate during rest, but a variation range is small. Therefore, when a variation tendency of an average value of the heart rate during rest for one week is calculated and shows a decreasing tendency even slightly, it can be determined that there is an improvement in the cardiopulmonary ability. As described above, the heart rate during rest is used as an index representing the cardiopulmonary ability that lasts for a long term as compared with the rising heart rate during exercise.

The physical strength level is an index indicating the physical strength level of the user U and is represented by a compared numerical value with an average physical strength level of the same age as 100. The physical strength level of the same age is derived from a statistical analysis of the basic physical strength information calculated based on the measurement data measured by a plurality of object persons of the same age. It is preferable that basic physical strength information related to a purpose of the user U is selected as the basic physical strength information used for the physical strength level. For example, in a case where the purpose is weight reduction, the maximum oxygen uptake representing the aerobic exercise ability may be the index. A complex index may be used using a plurality of pieces of basic physical strength information. It may be configured so as to calculate the index representing the basic physical strength using the plurality of pieces of basic physical strength information by selecting basic physical strength information to be used or changing weighting based on the purpose (for example, diet or exercise ability enhancement) of the user U.

When the basic habit information and the basic physical strength information of the user U are calculated, the calculation section 65 generates the basic information display screen D40 (FIG. 7) for notifying the user U. The basic information display screen D40 is generated by the server 40 and displayed on the information device 20.

FIG. 7 is a diagram showing an example of a basic information display screen. The basic information display screen D40 includes content of the basic habit information and the basic physical strength information of the user U and content for notifying the user U that a predetermined period is passed and the measurement of the measurement data for acquiring the basic information ends.

The basic information display screen D40 is configured to have date and time information D41, an end report D43, a physical strength level D44, calorie consumption D45, an average heart rate during exercise D46, an exercise time D47, an OK button D49, and the like.

The date and time information D41 is a region for displaying current date and time, and a date and time acquired from the clocking section of the device (for example, information device 20 and server 40) on which the basic information display screen D40 is displayed is displayed and updated every minute. A date and time when the basic information measurement ends may be displayed.

The end report D43 is a region for informing that the basic information measurement ends, and “Acquisition of basic information is completed” is displayed.

The physical strength level D44 is the physical strength level of the user U, and “88/100” is displayed. In the example, it is displayed that the physical strength level is a low 88 level for the physical strength level 100 of the same age.

The calorie consumption D45 is calorie consumption of the user U per week, and “2600 kcal/week” is displayed. The calorie consumption does not include the calorie consumption corresponding to a base metabolic rate.

The average heart rate during exercise D46 is the average heart rate during exercise of the user U, and “138 bpm” is displayed.

The exercise time D47 is an exercise time for each of the light exercise (zone 1) and the intense exercise (zone 2), and “43 minutes/day” in zone 1 and “120 minutes/week” in zone 2 are displayed.

The OK button D49 is a button for switching the display of the basic information display screen D40 to another screen.

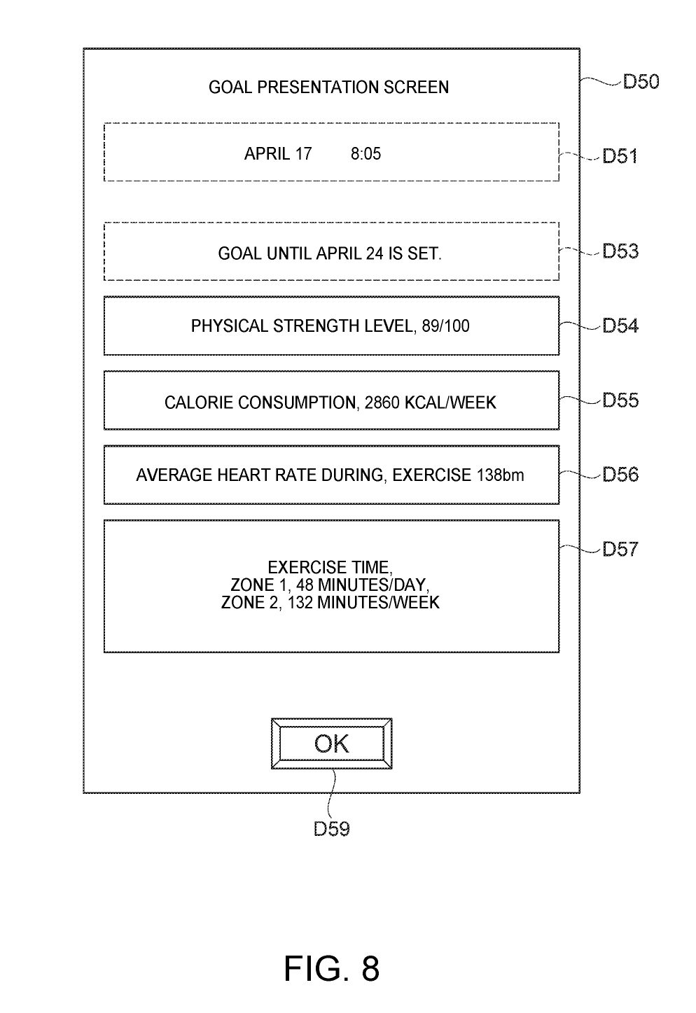

The goal setting section 69 determines the action plan of the user U with reference to the basic habit information and the basic physical strength information and generates a goal presentation screen D50 (FIG. 8) for notifying the user U of an immediate action goal. The goal presentation screen D50 is generated by the server 40, then approval processing (not shown) in which goal content is approved by the mentor in the mentor device 90 is performed, and is displayed on the information device 20. Alternatively, it may be configured such that the user U executes the approval processing and the result is informed to the mentor device 90. Functions of the goal setting section 69 are realized in processing S13 described above.

FIG. 8 is a diagram showing an example of a goal presentation screen.

The goal presentation screen D50 is configured to have date and time information D51, a goal report D53, a physical strength level D54, calorie consumption D55, an average heart rate during exercise D56, an exercise time D57, an OK button D59, and the like.

The date and time information D51 is a region for displaying current date and time, and a date and time acquired from the clocking section of the device (for example, information device 20, server 40, mentor device 90) on which the goal presentation screen D50 is displayed is displayed and updated every minute.

The goal report D53 is a report that the immediate goal is set, and “Goal until April 24 is set.” is displayed. It is preferable that the immediate goal is set in units of about one week.

The physical strength level D54 is a target value of the physical strength level of the user U, and “89/100” is displayed. In the example, the target value is increased by one level with respect to the value of the physical strength level D44 (FIG. 7).

The calorie consumption D55 is a target value of calorie consumption of the user U per week, and “2860 kcal/week” is displayed. In the example, the target value is obtained by multiplying the value of the calorie consumption D45 by a predetermined coefficient, for example, 1.1 times.

The average heart rate during exercise D56 is a target value of the average heart rate during exercise of the user U, and “138 bpm” is displayed. In the example, the target value is the same value as the average heart rate during exercise D46 and is the value not to raise an exercise load.

The exercise time D57 is a target time of the exercise time for each of the light exercise (zone 1) and the intense exercise (zone 2), and “48 minutes/day” in zone 1 and “132 minutes/week” in zone 2 are displayed. It is the target time in which an exercise time is increased by 5 minute/day in zone 1 and 12 minute/week in zone 2 than the exercise time D47.

The OK button D59 is a button for switching the display of the goal presentation screen D50 to another screen.

The storage section 70 is configured by a storage apparatus such as a read only memory (ROM) and a flash ROM, a random access memory (RAM), and a hard disk drive (HDD), and stores various programs for realizing each function section of the control section 50, data, and the like. A work area for temporarily storing data in processing of various types of processing, a variable value, a processing result, or the like is included. A database for managing a plurality of pieces of user information, mentor information, and the like managed in the server 40 is also included.

The storage section 70 stores a program 71, measurement data 73, screen data 75, basic data 80, and the like.

The program 71 is read and executed by the control section 50 to realize a function of each function section such as the acquisition section 51 included in the control section 50, the determination section 55, and the processing section 60. Processing of the program 71 will be described below using a flowchart diagram.

The measurement data 73 is data stored by the acquisition section 51, and information on the measurement time, the pulse rate, the calorie consumption, the exercise intensity, the calorie intake, and the like is stored for each measurement time, for example, in units of one second.

The screen data 75 is screen part data such as configuration information, buttons, and frames of display screens generated by the control section 50. Respective data of the advice display screen D10, the countdown display screen D20, the chat screen D30, the basic information display screen D40, the goal presentation screen D50, and the like generated using screen information generated by the generation section 61, the calculation section 65, and the goal setting section 69 are also stored.

The basic data 80 is data in which the basic information calculated by the calculation section 65 is stored, and is configured by basic habit data 81 and basic physical strength data 83.

In the basic habit data 81, the exercise time, the number of days of exercise, the calorie intake, the calorie consumption, and the like are stored.

In the basic physical strength data 83, the maximum oxygen uptake, the average heart rate during exercise, the rising heart rate during exercise, the heart rate during rest, and the like are stored.

### (Exercise Supporting Method)

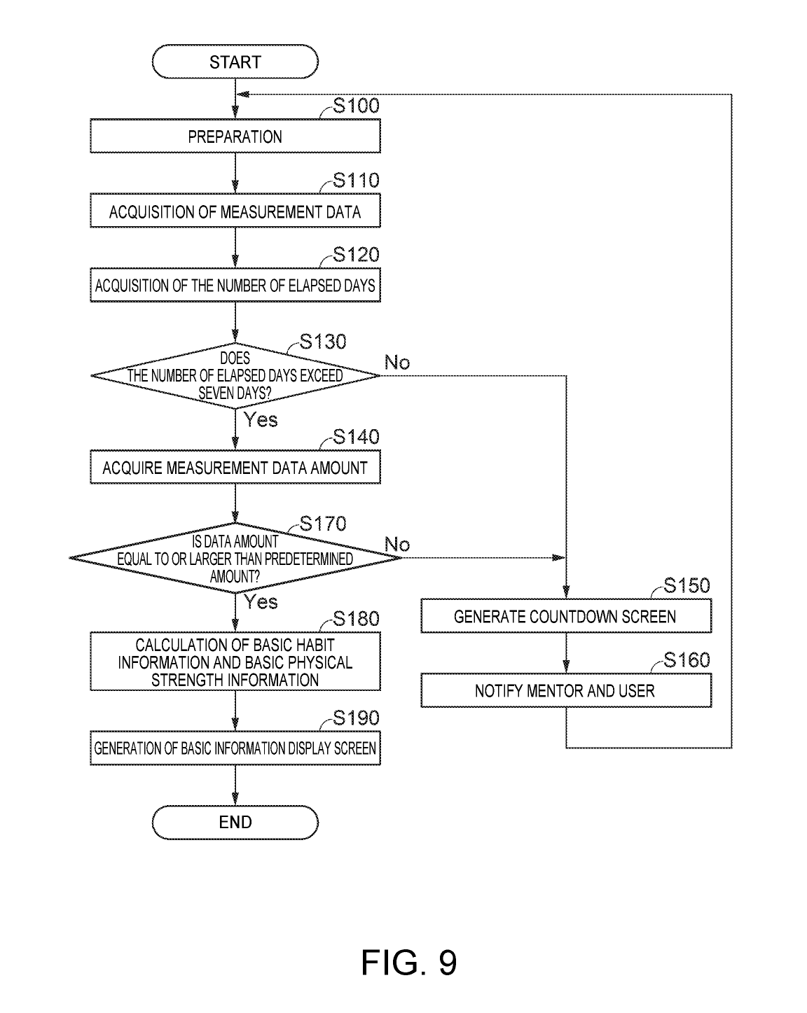

FIG. 9 is a flowchart diagram showing a flow of basic information acquisition processing. The flow shown in FIG. 9 is processing executed by the control section 50 by controlling each section such as the communication section 41, the clocking section 43, and the storage section 70 based on the program 71 stored in the storage section 70 of the server 40. The flow corresponds to an exercise supporting method, and the program 71 corresponds to a program.

In step S100, preparation necessary for processing each step is performed. For example, a timer is set using the real-time clock of the clocking section 43. The timer is used at least for counting the number of elapsed days and the like. The unique information, the purpose information, the service period information, and the like relating to the user U acquired by the user registration processing in processing S11 are taken into variables as the user information.

In step S110, the measurement data is acquired. Specifically, communication with the information device 20 is established through the communication section 41. The measurement data accumulated in the information device 20 is received. The received measurement data is stored in the storage section 70 (measurement data 73) in association with the user information acquired in step S100. The step corresponds to an acquisition step.

In step S120, the number of elapsed days is acquired. Specifically, the measurement time of the measurement data initially measured by the user U is acquired from the measurement data 73. Current time is acquired by the clocking section 43, the difference between the measurement time and current time is taken, and the number of elapsed days is calculated.

In step S130, it is determined whether the number of elapsed days exceeds seven days as the predetermined number of days. The processing proceeds to step S140 in a case where the number of elapsed days exceeds seven days (step S130; Yes), the processing proceeds to step S150 in a case where the number of elapsed days does not exceed seven days (step S130; No).

In step S140, a measurement data amount is acquired. Specifically, the measurement data 73 is read, and a sum of the measurement data for seven days is calculated.

In step S150, screen data of the countdown display screen is generated. A difference of the number of elapsed days from seven days is calculated, and the screen data of the countdown display screen D20 is generated.

In step S160, the mentor and the user are notified. Specifically, the user U and the mentor M are informed that the countdown display screen D20 is generated or the calculation of the basic information is completed. The user U and the mentor M may be informed by mail transmission, a pop-up screen using a web browser function, a monitoring function by a resident service program, or the like. Alternatively, informing is performed to prompt implementation of advice transmission (advice display screen D10) to the user U, an information exchange (chat screen D30) with the user U, and the like.

In step S170, it is determined whether the data amount is equal to or larger than the predetermined amount. Specifically, it is determined whether the measurement data amount acquired in step S140 is a data amount for seven days. The processing proceeds to step S180 in a case where the measurement data amount is equal to or larger than the data amount for seven days (step S170; Yes), the processing proceeds to step S150 in a case where the measurement data amount is less than the data amount for seven days (step S170; No). In the case where the processing proceeds to step S150, the number of days obtained by adding the number of days for measuring missing data amount is displayed in the countdown time D24 on the countdown display screen D20. The steps S130 and S170 correspond to a determination step.

In step S180, the basic habit information and the basic physical strength information are calculated. Specifically, the measurement data 73 is read, and respective data of the basic habit information and the basic physical strength information are calculated. The calculated data (basic data 80) is stored in the storage section 70.

In step S190, the basic information display screen is generated. The basic information display screen D40 is generated using the basic data 80.

The steps S150, S160, S180, and S190 correspond to a processing step.

When the flow ends, the processing proceeds from processing 512 (FIG. 2) to processing 513, and the determination of the action plan of the user U by the goal setting section 69 and the generation of the goal presentation screen D50 are performed.

As described above, in the exercise supporting system 1 according to the embodiment, it is possible to obtain the following effects.

The acquisition section 51 acquires the measurement data obtained by measuring the biological information and the activity information of the user U. The determination section 55 compares a measurement period with the predetermined number of days (seven days). In the case where the measurement period is equal to or less than seven days, the advice display screen D10, the countdown display screen D20, and the chat screen D30 notified to the user U by the generation section 61 (processing section 60) are generated. The user U can obtain information on the generated screen while measuring the measurement data. Since the countdown display screen D20 is generated at least once a day, there is a reduced possibility that the user U is in an uneasy state in which it is not known how long the measurement period lasts during the measurement period.

Since the pieces of information on the advice display screen D10 and the chat screen D30 are generated based on the measurement data updated each time it is acquired, it can be information in which a latest exercise situation of the user U is reflected. For example, in a case where there is a possibility that the user performs exercise far from usual, it is possible to generate screen information (advice display screen D10) that prompts the user to exercise as usual. Accordingly, it is possible to create an exercise plan based on the exercise situation of the user as usual.

The invention is not limited to the embodiment described above, and various modifications and improvements can be added to the embodiment described above. Modification examples will be described below.

### Modification Example 1

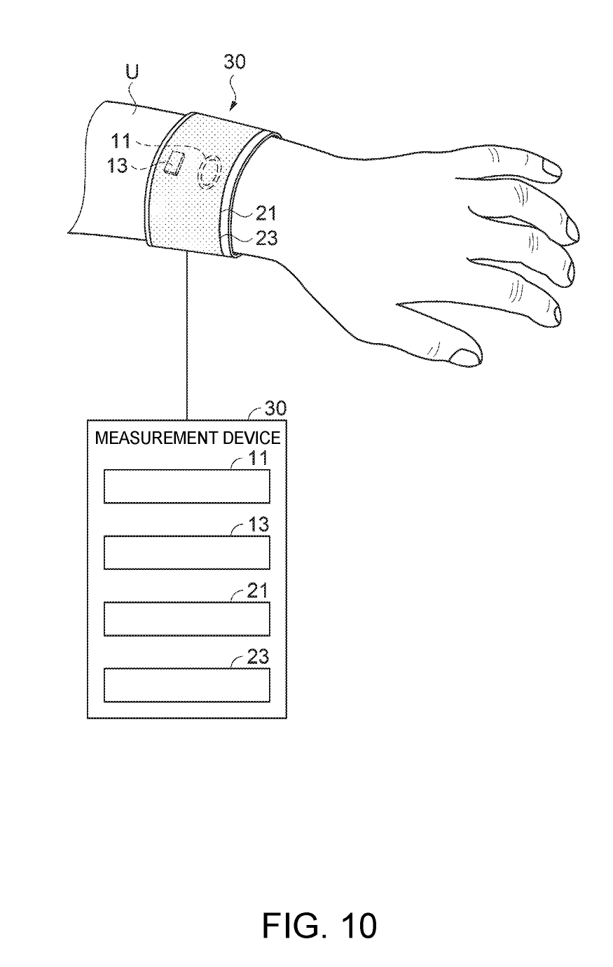

FIG. 10 is an explanatory diagram representing an outline of a measurement device according to a modification example.

In the embodiment described above, there is the configuration of a plurality of apparatuses including the measurement terminal 10 for measuring the biological information and the activity information of the user U and the information device 20 for displaying the screen information notified to the user U. However, the invention is not limited to the configuration, and a measurement device 30 as shown in FIG. 10 may be employed.

The measurement device 30 is a wristwatch type device worn on the arm or the like of the user U and is configured to have the measurement section 11, the output section 13, the display section 21, the operation section 23, and the like. The output section 13 has a communication function connected so as to be able to transmit and receive various data to and from the server 40 and the mentor device 90 through the communication 5 in the embodiment described above.

According to the configuration, it is possible for the user U to receive the exercise supporting service S1 only by wearing the measurement device 30. Since there is no need to use the plurality of apparatuses, it is easy to use and it is easy to provide an introductory guidance or the like on the exercise supporting service S1.

### Modification Example 2

In the embodiment and the modification example described above, the basic physical strength information is calculated from the measurement data measured by the habitual exercise as usual. However, the invention is not limited to such measurement data. Exercise content may be designated for the user U, and the measurement data of the designated exercise content may be acquired. For example, a 20-meter shuttle run, three-minute walking, or the like is designated as the exercise content. The maximum oxygen uptake of the user U can be estimated from the measurement data by the 20-meter shuttle run using “20-meter shuttle run maximum oxygen uptake estimation table” published by Ministry of Education, Culture, Sports, Science and Technology, and can be used as the basic physical strength information. In the three-minute walking, since the measurement data can be acquired in a short time of three minutes, it is possible to collect data of the same condition from many users. It is possible to accurately grasp the basic physical strength information and the physical strength level during or after the exercise supporting service S1 of the user U by statistically analyzing such data.

### Modification Example 3

In the embodiment and the modification examples described above, the exercise supporting system 1 includes the mentor device 90, but the invention is not limited to such a configuration. The mentor device 90 may not be included. In such a configuration, the server 40 may acquire the measurement data and automatically generates the advice wording for the user U based on an analyzed result. It is possible to configure the advice display screen D10 using the automatically generated advice wording and notify the user U.

### Modification Example 4

In the embodiment and the modification examples described above, in a case where the measurement period does not satisfy the predetermined number of days, the measurement data is acquired and the screen information notified to the user U is generated. However, further, the basic habit information and the basic physical strength information may be calculated from the measurement data acquired during the period. In a case where the accuracy of the basic habit information and the basic physical strength information increases during the acquisition of the measurement data, the acquisition of the measurement data may be stopped and the processing may proceed from processing S12 to processing S13 shown in FIG. 2. According to such a configuration, it is possible to deal with users having various exercise habits. For example, for a user who has a habit of doing intense exercise twice in three weeks, it is possible to accurately calculate the basic habit information of the user by setting the predetermined number of days to as long as one month (30 days).

There is probably a demand that the user U desires to immediately receive advice by the mentor M before completion of acquiring the basic information. In order to respond to the demand, “skip button” for interrupting the acquisition of the basic information may be displayed on the countdown display screen D20. In the case, when the user U selects the skip button on the measurement terminal 10 or the information device 20, stop information indicating that the skip button is selected is transmitted to the control section 50 of the server 40 from the measurement terminal 10 or the information device 20. The control section 50 of the server 40 informs the mentor device 90 of information indicating that the measurement of the basic information is omitted when receiving the stop information. The mentor device 90 determines whether the service can be started based on input information from the mentor M when receiving the informing. In a case where it is determined that the service can be started, informing for generating the basic information is transmitted from the mentor device 90 to the server 40 based on measurement data acquired until then, that is, measurement data in which a measurement period is less than the predetermined number of days. In a case where it is determined that the service cannot be started, informing for prompting to continue measuring the basic information is transmitted from the mentor device 90 to the information device 20 of the user U or the measurement terminal 10. Even though the accuracy of the basic information decreases by including such processing, it is possible to provide a service following a request of a user.

The control section 50 of the server 40 may add a configuration of supplementing and modifying the basic information with measurement data acquired during a period in which the user U receives the service according to the modes of the embodiment described above. It is possible to recover or improve the accuracy of the basic information by employing such a configuration.

### Modification Example 5

In the embodiment and the modification examples described above, the advice display screen D10, the countdown display screen D20, and the chat screen D30 are exemplified as the screen information generated in the case where the measurement period does not satisfy the predetermined number of days. However, the invention is not limited to the pieces of screen information, and it may be information such as guidance on a measurement state and an operation method. For example, in a state where measurement data cannot be measured from time to time, it may be information advising the user U about correct wearing method of the measurement terminal 10. It is possible to acquire more accurate measurement data by generating such information and providing the generated information to the user U.

### Modification Example 6

In the embodiment described above and the modification examples, the condition in the determination section 55 is described with respect to the two type conditions. However, the invention is not limited to the two type conditions described above as long as it is a determination condition necessary for calculating the basic habit information and the basic physical strength information. For example, the determination section 55 may determine the measurement period by any one of the two type conditions, or may be satisfied in a case where a time exceeds a predetermined time and may not be satisfied in a case where the time is equal to or less than the predetermined time. In a case where the user U is instructed so as to wear the measurement terminal 10 except during sleeping, a sum of time during awakening per day may be set as the predetermined time. For example, the predetermined time may be 112 hours (16 hours×seven days).

### Modification Example 7

In the embodiment and the modification examples described above, as the purpose of using the exercise supporting service S1, the weight loss of the body weight and body fat, or the increase in the physical strength of the user is described as the example, but the purpose to use may be a specific exercise ability improvement or the like. In such a case, the goal achievement state may be, for example, a time for running a predetermined distance such as 10 km or 20 km, a distance that can run continuously (run the whole distance), or the like. The physical strength index including the maximum oxygen uptake, exercise duration up to the maximum pulse rate, a lung capacity, an index of endurance, and the like may be set as the target value.

### Modification Example 8

In the embodiment and the modification examples described above, the example in which the communication between the mentor M and the user U is performed on the advice display screen D10 and the chat screen D30 in step S160 (FIG. 9) is described, but the invention is not limited to the timings. The communication between the mentor M and the user U may be performed at an arbitrary timing according to their intention or it may be configured to prompt the communication by providing a trigger at an arbitrary timing (for example, before sleeping or after getting up of the user U) from the server 40. As the timing of providing the trigger from the server 40 to the mentor M (transmitting trigger signal), it may be the time when the user U completes the registration processing to the service in processing S11, the time when the acquisition of the basic information of the user U is completed in processing S12, or the time when the action plan of the user U is set in processing S13.

A trigger signal may be transmitted according to a change in an action implementation situation of the user U and progress of the goal achievement of the user U. In the case, for example, as the change in the action implementation situation of the user U, for example, in a case where the control section 50 determines that the difference between date and time information of the measurement data transmitted from the measurement terminal 10 to the server 40 and current date and time information is equal to or larger than a predetermined period, and in a case where the control section 50 of the server 40 determines that the action implementation of the user U deviates from a plan as a result of comparison between an analysis result of the measurement data and plan information, the trigger signal is transmitted from the server 40 to the mentor device 90.

### Modification Example 9

The processing of lending the measurement terminal 10 to the user U in processing S11 of the exercise supporting service S1 (FIG. 2) exemplified in the embodiment described above and the modification examples will be described in detail. In the exercise supporting service S1, a course of lending the measurement terminal 10 to the user U during the service period is prepared. When the user U selects such a lending course, the control section 50 of the server 40 sets deadline information including a return date of a lent article based on the service period information. The deadline information is transmitted to the measurement terminal 10 used by the user U, the information device 20, the mentor device 90, and the like, and is stored in each device.

In the measurement terminal 10, the information device 20, and the mentor device 90, the deadline information is compared with the current date and time, and when a predetermined condition is satisfied, the user U is informed that the return day is approaching. In the measurement terminal 10, in a case where a return deadline is exceeded, a part or all functions of the measurement terminal 10 may be stopped. Information that prompts an extension of the service period (contract period) and an extension of a lending period may be provided without stopping the functions.

### Modification Example 10

In the embodiment described above and the modification examples, in the repeat R12 of the exercise supporting service S1 (FIG. 2), the first type condition exemplified in the determination section 55 (FIG. 3), and step S130 of the flowchart diagram (FIG. 9), the measurement is continued by setting the predetermined number of days from the measurement start by the measurement terminal 10 to seven days as a preferable example, but the invention is not limited to such a configuration. For example, in a case where the user U already uses the measurement terminal 10 before applying for the exercise supporting service S1 and valid measurement data 73 is accumulated in the server 40, it is possible to set the number of days obtained by subtracting the number of days of the accumulated measurement data 73 from seven days as the predetermined number of days. For example, in a case where the measurement data 73 for three days of Monday, Tuesday, and Wednesday is accumulated already before the service application, the predetermined number of days after the measurement start may be four days of Thursday, Friday, Sunday, and Sunday.

In such a case, in a state where “skip button” is provided in the countdown display screen D20 (FIG. 5) and the predetermined number of days is kept for seven days, when four days are passed after the measurement start, the skip button may be displayed or it may be changed from an invalid state to a valid state. Since a case where the skip button is ready to be pressed means that a necessary amount of the basic information is acquired, the user U can press the skip button and end the measurement of the basic information.

### REFERENCE SIGNS LIST

1: exercise supporting system
3: communication
5: communication
10: measurement terminal
11: measurement section
13: output section
20: information device
21: display section
23: operation section
30: measurement device
40: server
41: communication section
43: clocking section
50: control section
51: acquisition section
55: determination section
60: processing section
61: generation section
65: calculation section
69: goal setting section
70: storage section
71: program
73: measurement data
75: screen data
80: basic data
81: basic habit data
83: basic physical strength data
90: mentor device
91: editing section
S1: exercise supporting service
D10: advice display screen
D20: countdown display screen
D30: chat screen
D40: basic information display screen
D50: goal presentation screen.

1. An exercise assistance system comprising:
an acquisition section that acquires measurement data including at least one of biological information and activity information of a user;
a determination section that determines whether a measurement period of the measurement data satisfies a predetermined condition; and
a processing section that calculates basic habit information and basic physical strength information based on the measurement data in a case where the determination section satisfies the predetermined condition, and generates first information to notify the user based on the measurement data in a case where the determination section does not satisfy the predetermined condition.

2. The exercise assistance system according to claim 1,
wherein the predetermined condition is satisfied in a case where the measurement period exceeds the predetermined number of days and is not satisfied in a case where the measurement period is equal to or less than the predetermined number of days.

3. The exercise assistance system according to claim 1,
wherein the first information includes at least one of an advice display screen, a chat screen, and a countdown display screen.

4. The exercise assistance system according to claim 1,
wherein the determination section further compares a data amount of the measurement data with a predetermined amount in the case where the predetermined condition is satisfied, and the predetermined condition is satisfied in a case where the data amount is equal to or larger than the predetermined amount and the predetermined condition is not satisfied in a case where the data amount is less than the predetermined amount.

5. The exercise assistance system according to claim 1,
wherein the basic habit information includes at least one of an exercise time, the number of days of exercise, calorie intake, and calorie consumption.

6. The exercise assistance system according to claim 5,
wherein the basic habit information is an average value or a sum value per certain period in the measurement period.

7. The exercise assistance system according to claim 1,
wherein the basic physical strength information includes at least one of a physical strength level, maximum oxygen uptake, an average heart rate during exercise, a rising heart rate during exercise, and a heart rate during rest.

8. The exercise assistance system according to claim 1, further comprising:
a measurement terminal including
a measurement section that measures the measurement data of the user, and
an output section that outputs the measurement data to the acquisition section.

9. An information device comprising:
a display section that displays the first information generated by the processing section of the exercise assistance system according to claim 1.

10. A measurement device comprising:
an output section that outputs the measurement data to the acquisition section of the exercise assistance system according to claim 1;
a measurement section that measures the measurement data of the user, and
a display section that displays the first information generated by the processing section.

11. An exercise assistance method comprising:
an acquisition step of acquiring measurement data including at least one of biological information and activity information of a user;
a determination step of determining whether a measurement period of the measurement data satisfies a predetermined condition; and
a processing step of calculating basic habit information and basic physical strength information based on the measurement data in a case where the determination step satisfies the predetermined condition, and generating first information to notify the user based on the measurement data in a case where the determination step does not satisfy the predetermined condition.

12. The exercise assistance method according to claim 11,
wherein the predetermined condition is satisfied in a case where the measurement period exceeds the predetermined number of days and is not satisfied in a case where the measurement period is equal to or less than the predetermined number of days.

13. The exercise assistance method according to claim 11,
wherein the first information includes at least one of an advice display screen, a chat screen, and a countdown display screen.

14. The exercise assistance method according to claim 11,
wherein in the determination step, a data amount of the measurement data is further compared with a predetermined amount in the case where the predetermined condition is satisfied, and the predetermined condition is satisfied in a case where the data amount is equal to or larger than the predetermined amount and the predetermined condition is not satisfied in a case where the data amount is less than the predetermined amount.

15. The exercise assistance method according to claim 11,
wherein the basic habit information includes at least one of an exercise time, the number of days of exercise, calorie intake, and calorie consumption.

16. The exercise assistance method according to claim 15,
wherein the basic habit information is an average value or a sum value per certain period in the measurement period.

17. The exercise assistance system according to claim 11,
wherein the basic physical strength information includes at least one of a physical strength level, maximum oxygen uptake, an average heart rate during exercise, a rising heart rate during exercise, and a heart rate during rest.

18. A program that causes a computer to execute an exercise assisting method, the method comprising:
an acquisition step of acquiring measurement data including at least one of biological information and activity information of a user;
a determination step of determining whether a measurement period of the measurement data satisfies a predetermined condition; and
a processing step of calculating basic habit information and basic physical strength information based on the measurement data in a case where the determination step satisfies the predetermined condition, and generating first information to notify the user based on the measurement data in a case where the determination step does not satisfy the predetermined condition.
